(19) United States
(12) Patent Application Publication
(10) Pub. No.: US 2020/0221997 A1
(43) Pub. Date: Jul. 16, 2020

(54) SMART MEDICATION ALERT AND DELIVERY SYSTEM AND METHOD THEREOF

(71) Applicant: Winston Therapeutics, Inc., Spokane, WA (US)

(72) Inventors: Thomas Beck, Spokane, WA (US); Wendy Popa, Spokane, WA (US)

(73) Assignee: Winston Therapeutics, Inc.

(21) Appl. No.: 16/739,586

(22) Filed: Jan. 10, 2020

Related U.S. Application Data
(60) Provisional application No. 62/790,955, filed on Jan. 10, 2019.

(51) Int. Cl. A61B 5/00; G16H 20/13; A61J 7/00; G08B 23/00; G08B 7/06

(52) U.S. Cl. CPC A61B 5/4839; G16H 20/13; A61B 5/746; A61B 2560/0242; G08B 23/00; G08B 7/06; A61J 7/0076

(57) ABSTRACT

A digital therapeutic treatment or intervention may include a device to alert the user to a need situation related to the user's health status and indicate to the user a treatment or intervention recommendation according to applicable signs and/or symptoms detected and/or known and/or calculated based on information received by the device from the user. The treatment or intervention may include alerting the user to the need to take a specific medication of a specific strength/dosage. Additionally, and/or alternatively, the treatment or intervention may include activating a smart medication delivery device to automatically deliver or release into/onto the user's body a specific medication of a specific strength/dosage.

### CROSS REFERENCE TO RELATED APPLICATIONS

This application claims priority to U.S. Provisional Patent Application No. 62/790,955, filed on Jan. 10, 2019, entitled “SMART MEDICATION ALERT AND DELIVERY SYSTEM AND METHOD THEREOF,” the contents of which are herein incorporated by reference.

### BACKGROUND

Recently, an industry has emerged referred to as digital therapeutics. Digital therapeutics has been described as a health discipline and treatment option that utilizes a digital and often online health technologies to treat a medical or psychological condition. The treatment relies on behavioral and lifestyle changes usually spurred by a collection of digital impetuses. In other words, digital therapeutics intend to provide a way to deliver evidence-based therapeutic interventions to patients, which interventions are driven by software programs to prevent, manage, or treat a medical disorder or disease. Digital therapeutics are used independently or in concert with medications, devices, or other therapies to optimize patient care and health outcomes. Digital therapeutics products incorporate advanced technology best practices relating to design, clinical validation, usability, and data security. Further, digital therapeutics may be validated by regulatory bodies as required to support product claims regarding risk, efficacy, and intended use.

Currently, when a patient is diagnosed with a health condition, frequently a health professional may desire to recommend to the user a treatment/intervention, such as a prescription of medication, in an attempt to address the health condition of the user. Due to the biological and environmental variances between any two patients, it is not uncommon that a first treatment/intervention, such as a first medication of a first dosage amount or strength when applied at a predetermined timing, that works for a first patient, may be totally or partially ineffective for a second patient for whom the first treatment/intervention is recommended. In some instances, the treatment/intervention may be effective (or more effective) when applied at a specific time of the patient's day, such as upon waking in the morning, while if taken later after a certain activity, it may be ineffective (or less effective). Alternatively, the treatment/intervention may be effective if given at a first dosage amount or strength in the morning, but a distinct increased or decreased second dosage amount or strength is better later in the day. However, it is cumbersome, if allowed or even possible, for a patient (and the health professional) to have to determine and remember the precise increased/decreased amount/strength of the second dosage to take later in the day.

Thus, a better smarter manner of determining and delivering the adequate treatment/intervention for a specific health status that may vary not only throughout a given day, but also from day to day, is desired.

### BRIEF DESCRIPTION OF THE DRAWINGS

The Detailed Description is set forth with reference to the accompanying FIGURES. In the FIGURES, the left-most digit(s) of a reference number identifies the FIGURE in which the reference number first appears. The use of the same reference numbers in different FIGURES indicates similar or identical items. Furthermore, the drawings may be considered as providing an approximate depiction of the relative sizes of the individual components within individual FIGURES. However, the drawings are not to scale, and the relative sizes of the individual components, both within individual FIGURES and between the different FIGURES, may vary from what is depicted. In particular, some of the FIGURES may depict components as a certain size or shape, while other FIGURES may depict the same components on a larger scale or differently shaped for the sake of clarity.

FIG. 1 schematically represents a user with an embodiment of a user device to assist with the digital therapeutic according to the instant disclosure.

### DETAILED DESCRIPTION

### Overview

This disclosure is directed to embodiments of a digital therapeutic treatment. Further, a treatment or intervention according to the instant disclosure may include a device to alert the user to a need situation related to the user's health status and indicate to the user a treatment or intervention recommendation according to applicable signs and/or symptoms detected and/or known and/or calculated based on information received by the device from the user. The treatment or intervention may include alerting the user to the need to take a specific medication of a specific strength/dosage. Additionally, and/or alternatively, the treatment or intervention may include activating a smart medication delivery device to automatically deliver or release into/onto the user's body a specific medication of a specific strength/dosage.

In an embodiment, the device may determine user needs and the associated treatment or intervention corresponding to a user's health status based on data such as detected biological information, detected/retrieved situational/environmental conditions, and/or entered input to the device from the user or a third party caring for the user. Regardless of the data intake method, the device may determine a potential treatment or intervention to address the user's need situation corresponding to the user's health status using one or more processors configured to execute instructions stored on memory, which instructions when executed cause the processors to perform steps. The steps may include receiving data, processing the data, and alerting the user to perform the potential treatment or intervention, and/or communicating with and activating a smart medication delivery device to automatically deliver or release (or apply) the potential treatment or intervention into/onto the user's body.

In an embodiment, the device may be a computing device including the one or more processors needed to process the data received. Additionally, and/or alternatively, the device may be a computing device having sensors that detect biological data of the user and/or environmental data surrounding the user. In an embodiment, the device may include a wired or wireless data receiving and transmitting component via which the device may transmit the data received to a secondary computing device having one or more processors that are configured to process the received data and determine the potential treatment or intervention for the user. The secondary computing device may also have a data receiving/transmitting component with which to send a signal to the device indicating instructions related to the determined potential treatment or intervention for the user. In an embodiment, the device may further include an indicator component to alert the user to perform the potential treatment or intervention. Additionally, and/or alternatively, the device may include an indicator component to activate the device to relay instructions to a smart medication delivery device to automatically execute the instructions of the potential treatment or intervention.

In an embodiment, the device may be a wearable device for the user to wear on the user's body (e.g., a watch, a surgically attached (internally or externally) electronic device, a bracelet, a necklace, a hat, a hearing-assistance device, a sensor-embedded article of clothing, glasses, etc.). Alternatively, the device may be a portable device (e.g., a phone, a tablet, etc.) that the user may carry about the user's body (e.g., in a pocket, in a case, in a backpack, in a purse/carryall bag, etc.), and the device may have a sensor component to detect biological and/or environmental data.

The device may be configured to detect characteristics of the user's body and/or receive data characteristics of the user's body from a smart medication delivery device. The characteristics of the user's body to be detected or received may include: cardiovascular system data, pulmonary system data, digestive system data, nervous system data, immune system data, exocrine system data, excretory system data, endocrine system data, etc. Some specific embodiments of system data may include: pulse, pulse rate, blood pressure, sweat, sweat rate, sodium balance, blood contents, cellular biological content/quality factors, lung function, oxygen levels, etc.

Based on the data of the characteristics of the user's body detected, determined, or received, the device may determine a user health status. Upon a determination of the user health status, the status may be compared to health status standards according to the age/situation/biological preconditions corresponding to the specific user. If the user's health status exceeds or does not meet predetermined health status thresholds (maximums and/or minimums) considered to indicate a preferred health status for the specific user, the device is configured to execute the associated instructions (e.g., alert the user to the situation and provide further instructions to address the need situation, activate the device or a smart medication delivery device to automatically deliver or release the potential treatment or intervention into/onto the user's body.

In an embodiment, the smart medication delivery device may include one or more of: a surgically implantable device, an ingestible device, an orally receivable device, a dermally applied device, an injectable device, etc. Examples of the smart medication delivery device may include skin patches, pills, pumps, catheter delivery devices, etc., and such devices may be electro-mechanical devices or may include electro-mechanical components to be activated and cause delivery or release of specific dosages of medications that are targeted to address the specific and instant health status of the user of the devices (the computing device and/or the smart medication delivery device).

In an embodiment, the device may be configured to allow a user to delay or completely prevent delivery or release of a treatment or intervention by the smart medication delivery device upon alert from the device that a treatment or intervention is suggested or about to be delivered or released into/onto the user's body.

In an embodiment, the treatment or intervention to be delivered or released into/onto the user's body may include a user-specific-customized dosage/strength of one or more medications/treatments. Additionally, and/or alternatively, the treatment or intervention to be delivered or released into/onto the user's body may include a user-specific-customized timed introduction of one or more medications/treatments.

### Illustrative Embodiments of the Devices of the Instant Disclosure

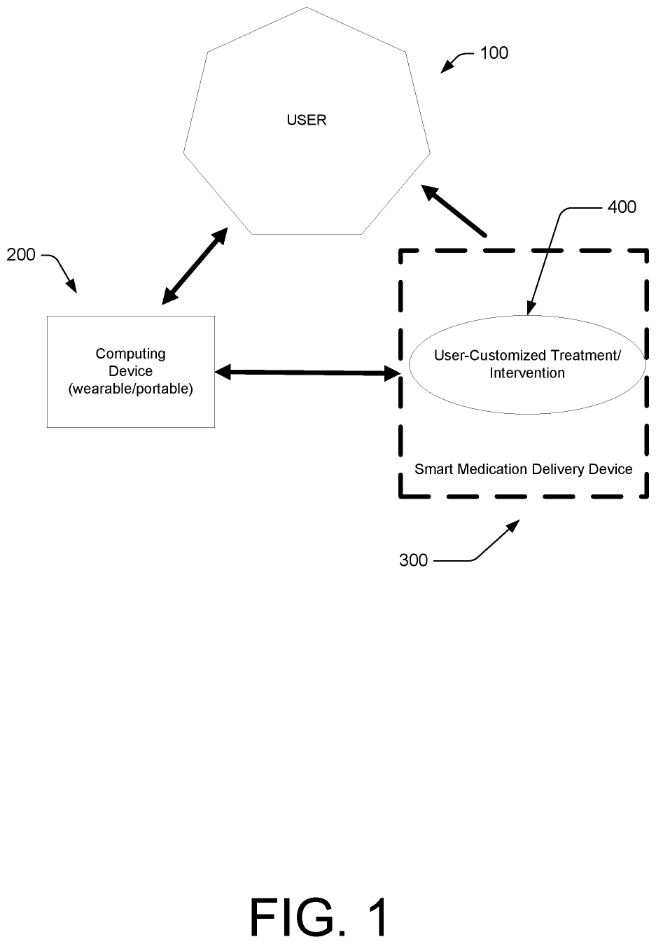

Each of the example implementations below may be represented by the illustration of FIG. 1. As illustrated in FIG. 1, the heptagon represents a user 100; the small rectangle represents the computing device 200 that may be a computing device such as a wearable or portable device including one or more of: one or more sensors, one or more processors, memory (data bank), data transmission/receiving components, or one or more signal and/or alert transmitter/indicator components (e.g., light, sound, display interface, etc.); the large (dashed-line) square represents the optional (hence the dashed-line) smart medication delivery system 300; and the oval represents the user-customized treatment/intervention 400. In an embodiment, the computing device 200 may receive or detect biological data from the user 100 and/or environmental data in the environment of the user 100. Further, the computing device may alert or otherwise indicate to the user the recommended treatment/intervention to occur or be delivered/released. Thus, the arrow between the user 100 and the computing device 200 indicates input/detection/information flow in both directions. Similarly, the arrow between the computing device 200 and the smart medication delivery system 300 indicates information/communication flow in both directions, to and from, transmission/receipt/confirmation. Note, however that whether the user 100 is alerted to the need situation and executes the application/intake of the treatment/intervention 400 without assistance from a smart medication delivery device, or if the treatment/intervention 400 is supplied via the smart medication delivery device, the arrow indicates that the direction of the treatment/intervention 400 only flows in one direction—to the user 100.

In an example implementation of the instant disclosure, the following scenario describes the possibilities attainable with the devices described herein. A first user and a second user (not depicted) may each have a portable or wearable device, such as a phone or watch, configured to monitor cardiovascular system data of the users, respectively. The first user and the second user may also carry with them pills of varying dosage amounts or strengths of a particular medication intended to address a health condition of the respective users for which the users carry the pills. The portable or wearable devices, respectively, may periodically monitor or automatically detect biological/environmental data, or the user may be prompted to execute an action with the device so as to receive data to determine the respective users' health status. Upon determination of the health status of the first user, the device may alert the first user to a need situation and instruct the first user to intake a first particular treatment or intervention, such as a first particular dosage amount and/or strength of the medication and/or at a predetermined timing that is customized to address the first user in a way that is especially effective for the first user. Likewise, upon determination of the health status of the second user, the device may alert the second user to a need situation and instruct the second user to intake a second particular treatment or intervention, such as a second particular dosage amount and/or strength of the medication and/or at a predetermined timing that is customized to address the second user in a way that is especially effective for the second user. An advantage of the instant disclosure is that the first particular dosage and the second particular dosage may be different and are customized for the body functions of the specific users.

In another example implementation of the instant disclosure, the following scenario describes the possibilities attainable with the devices described herein. A first user and a second user may each have a portable or wearable device, such as a phone or watch, configured to monitor cardiovascular system data of the users, respectively. The first user and the second user may also have ingested a smart medication delivery device, such as an electro-mechanical, biologically-safe pill. The pills may contain several dosages of varying amounts and/or strengths of a medication intended to address a health condition of the respective users for which the users have ingested the pills. The pills may be further configured to receive communication from the wearable device. The pills may be further configured such that, upon receipt of the communication from the wearable devices, respectively, the electro-mechanical componentry of the pill taken by the first user may release a first particular dosage amount and/or strength of the medication and/or at a predetermined timing that is customized to address the first user in a way that is especially effective for the first user; and the electro-mechanical componentry of the pill taken by the second user may release a second particular dosage amount and/or strength of the medication and/or at a predetermined timing that is customized to address the second user in a way that is especially effective for the second user. An advantage of the instant disclosure is that the first particular dosage and the second particular dosage may be different and are customized for the body functions of the specific users.

A further aspect of the above implementation includes an embodiment of communication between the device and the smart medication delivery device. For example, in an embodiment, the device may be a phone or watch that communicates to the ingested/implanted/adhered/injected smart medication delivery device via one or more distinct frequencies of sound to indicate which dosage amount/strength to release to the user. That is, a first frequency may cause the delivery device to release a first dosage amount or strength; and a second frequency, distinct from the first frequency, may cause the delivery device to release a second dosage amount or strength that is distinct from the first dosage amount or strength.

### Embodiments of Hardware/Software for Implementation in Computing Devices

The specific processor(s), memory, and other signal/data transmission componentry, hardware, software, and other electronic components are those that may be familiar for standard computing/processing/signal and data transmission purposes as are common in the industry. For a non-limiting example, a wearable device may be a smart watch using application software to activate alerts and initiate signals.

### CONCLUSION

Although several embodiments have been described in language specific to structural features and/or methodological acts, it is to be understood that the claims are not necessarily limited to the specific features or acts described. Rather, the specific features and acts are disclosed as illustrative forms of implementing the claimed subject matter.

What is claimed is:

1. A smart treatment/delivery system, comprising:
hardware and/or software configured to detect and/or receive biological and/or environmental data related to or directly from a user;
one or more processors configured to:
determine a health status of the user, and
alert the user and/or activate a smart medication delivery device to automatically deliver or release a determined dosage amount or strength of a treatment/intervention to the user, based at least in part on the detected and/or received biological and/or environmental data.
